(19) United States
(12) Patent Application Publication
(10) Pub. No.: US 2025/0359833 A1
(43) Pub. Date: Nov. 27, 2025

(54) PHOTON COUNTING CT APPARATUS

(71) Applicant: CANON MEDICAL SYSTEMS CORPORATION, Tochigi (JP)

(72) Inventors: Ryosuke HIGASHI, Nasushiobara (JP); Masao YAMAHANA, Nasushiobara (JP); Terutada SHUTO, Otawara (JP); Junichiro ARAOKA, Nasushiobara (JP)

(73) Assignee: CANON MEDICAL SYSTEMS CORPORATION, Tochigi (JP)

(21) Appl. No.: 19/214,587

(22) Filed: May 21, 2025

(30) Foreign Application Priority Data
May 22, 2024 (JP) 2024-083561

(51) Int. Cl. A61B 6/42; A61B 6/00; A61B 6/03; A61B 6/40; G16H 40/63

(52) U.S. Cl. CPC A61B 6/4241; A61B 6/03; A61B 6/4078; A61B 6/54; G16H 40/63

(57) ABSTRACT

According to one embodiment, a photon counting computed tomography (CT) apparatus includes an X-ray generator configured to generate an X-ray, an X-ray detector of a photon counting type configured to detect the X-ray that has passed through a subject, and processing circuitry configured to acquire count data by counting the number of X-ray photons for each energy band, based on a detection result of the X-ray by the X-ray detector, set a first condition and a second condition as band settings that are settings of the energy band, and execute successively a first scan that is performed under the first condition, and a second scan that is performed under the second condition, on the subject placed on a tabletop.

### CROSS-REFERENCE TO RELATED APPLICATIONS

This application is based upon and claims the benefit of priority from Japanese Patent Application No. 2024-083561, filed May 22, 2024, the entire contents of which are incorporated herein by reference.

### FIELD

Embodiments described herein relate generally to a photon counting computed tomography (CT) apparatus.

### BACKGROUND

Photon counting type X-ray detectors are known. In an examination using a photon counting type X-ray detector, count data that indicates X-ray intensity for each energy band (bin) is acquired. The count data is processed to generate various data, such as a material decomposition image.

The more the energy bands are set in acquiring count data using a photon counting type X-ray detector, the more various data to be generated in subsequent processing is available. However, a data size of the count data increases with an increase in the number of set energy bands, which consequently increases a load related to data transmission and storage.

### BRIEF DESCRIPTION OF THE DRAWINGS

FIG. 1 is a block diagram illustrating an example of a configuration of a photon counting computed tomography (CT) apparatus according to a first exemplary embodiment;

FIG. 2 is a flowchart illustrating a series of processing procedure of the photon counting CT apparatus according to the first exemplary embodiment;

FIG. 3 illustrates a setting example of a first condition and a second condition according to the first exemplary embodiment;

FIG. 4 illustrates another setting example of a first condition and a second condition according to the first exemplary embodiment; and

FIG. 5 illustrates an example of a band setting according to a third exemplary embodiment.

### DETAILED DESCRIPTION

According to one embodiment, a photon counting computed tomography (CT) apparatus includes an X-ray generator configured to generate an X-ray, an X-ray detector of a photon counting type configured to detect the X-ray that has passed through a subject, and processing circuitry configured to acquire count data by counting the number of X-ray photons for each energy band, based on a detection result of the X-ray by the X-ray detector, set a first condition and a second condition as band settings that are settings of the energy band, and execute successively a first scan that is performed under the first condition, and a second scan that is performed under the second condition, on the subject placed on a tabletop.

Various Embodiments will be described hereinafter with reference to the accompanying drawings.

### First Exemplary Embodiment

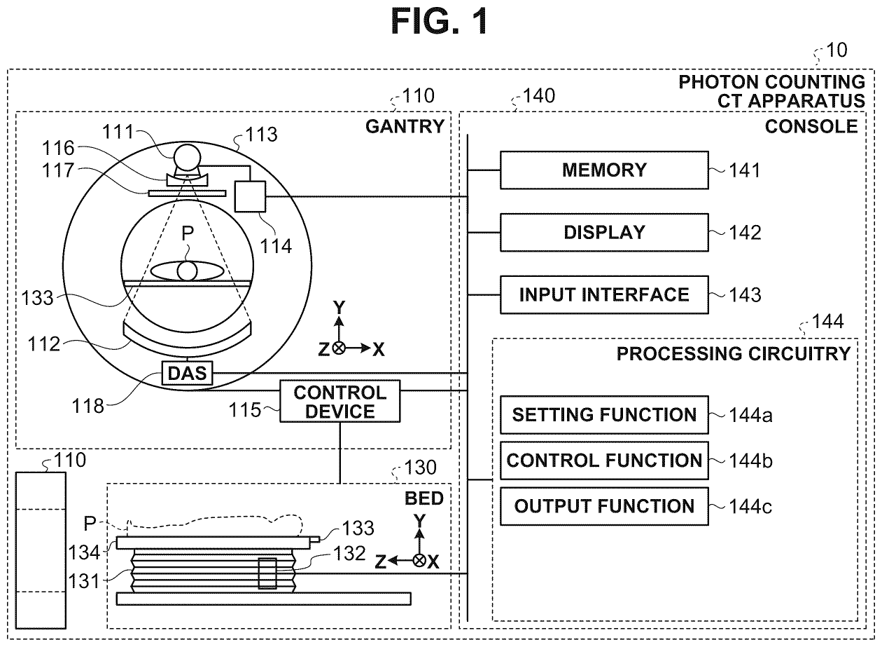

A photon counting computed tomography (PCCT) apparatus 10 illustrated in FIG. 1 is described as an example. The photon counting CT apparatus 10 is a type of an X-ray CT apparatus and includes an X-ray detector 112 of a photon counting type. The photon counting CT apparatus 10 includes a gantry 110, a bed 130, and a console 140, for example.

In FIG. 1, a rotation axis of a rotation frame 113 in a non-tilt state or a longitudinal direction of a tabletop 133 of the bed 130 is defined as a Z-axis direction. An axis direction perpendicular to the Z-axis direction and horizontal to a floor surface is defined as an X-axis direction. Further, an axis direction perpendicular to the Z-axis direction and vertical to the floor surface is defined as a Y-axis direction. FIG. 1 illustrates the gantry 110 from a plurality of directions for the sake of explanation, and illustrates a case where the photon counting CT apparatus 10 includes one gantry 110.

The gantry 110 includes an X-ray tube 111, the X-ray detector 112, the rotation frame 113, an X-ray high voltage device 114, a control device 115, a wedge 116, a collimator 117, and a data acquisition system (DAS) 118.

The X-ray tube 111 is a vacuum tube that includes a cathode (filament) that generates thermal electrons and an anode (target) that generates X-rays in response to collision of the thermal electrons. In response to application of a high voltage from the X-ray high voltage device 114, the X-ray tube 111 emits thermal electrons from the cathode to the anode, whereby X-rays to be emitted to a subject P are generated. The X-ray tube 111 is an example of an X-ray generator.

The X-ray detector 112 is a photon counting type X-ray detector and outputs, each time an X-ray photon is incident on the X-ray detector 112, a signal that is to be used to measure an energy value of the X-ray photon. The X-ray photon that is incident on the X-ray detector 112 has been emitted from the X-ray tube 111 and has passed through the subject P. The X-ray detector 112 includes a plurality of detection elements that output one pulse of an electrical signal (analog signal) each time the X-ray photon is incident thereon. By counting the number of electrical signals (pulses), the number of X-ray photons incident on each detection element is counted. Further, predetermined arithmetic processing on the signal is performed to measure an energy value of the X-ray photon that causes the output of the signal. For example, the X-ray detector 112 is an area detector in which a plurality of detection elements is arranged in a channel direction and a slice direction.

The above-described detection element is configured with, for example, a scintillator and a photosensor, such as a photomultiplier tube. In this case, the X-ray detector 112 is an indirect conversion type detector that converts the X-ray photon incident thereon into scintillator light by the scintillator and converts the scintillator light into an electrical signal by the photosensor, such as a photomultiplier tube. As another example, the above-described detection element is a semiconductor detection element, such as cadmium telluride (CdTe) or cadmium zinc telluride (CdZnTe), with an electrode disposed thereon. In this case, the X-ray detector 112 is a direct conversion type detector that directly converts the X-ray photon incident thereon into an electrical signal.

The rotation frame 113 is an annular frame that supports the X-ray tube 111 and the X-ray detector 112 in positions facing each other and rotates the X-ray tube 111 and the X-ray detector 112 by the control device 115. For example, the rotation frame 113 is a casting made of aluminum. The rotation frame 113 further supports the X-ray high voltage device 114, the wedge 116, the collimator 117, the DAS 118, and the like in addition to the X-ray tube 111 and the X-ray detector 112. In addition, the rotation frame 113 further supports various components, which are not illustrated in FIG. 1. In the following description, the rotation frame 113 and a part that rotates and moves together with the rotation frame 113 in the gantry 110 are also referred to as a rotation part.

The X-ray high voltage device 114 includes electrical circuitry, such as a transformer and a rectifier, a high voltage generation device that generates a high voltage to be applied to the X-ray tube 111, and an X-ray control device that controls an output voltage in accordance with an X-ray generated by the X-ray tube 111. The high voltage generation device may be a transformer type or an inverter type. The X-ray high voltage device 114 may be provided in the rotation frame 113 or in a fixing frame, which is not illustrated.

The control device 115 includes a processing circuitry that includes a central processing unit (CPU) and a driving mechanism, such as a motor and an actuator. The control device 115 receives an input signal from an input interface 143 and controls operations of the gantry 110 and the bed 130. For example, the control device 115 controls rotation of the rotation frame 113, tilting of the gantry 110, and operations of the bed 130 and the tabletop 133. The control device 115 may be provided in the gantry 110 or in the console 140.

The wedge 116 is a filter that adjusts an amount of X-rays emitted from the X-ray tube 111. Specifically, the wedge 116 is a filter that transmits and attenuates the X-rays emitted from the X-ray tube 111 in such a manner that the X-rays emitted from the X-ray tube 111 to the subject P have a predetermined distribution. For example, the wedge 116 is a wedge filter or a bow-tie filter, and is a filter made of aluminum or other material to have a predetermined target angle and thickness.

The collimator 117 includes a lead plate or the like to narrow down an irradiation range of the X-rays which has been passed through the wedge 116, and has a slit formed by a combination of a plurality of the lead plates or the like. The collimator 117 may also be referred to as an X-ray diaphragm. FIG. 1 illustrates a case where the wedge 116 is arranged between the X-ray tube 111 and the collimator 117, but the collimator 117 may be arranged between the X-ray tube 111 and the wedge 116. In this case, the wedge 116 transmits and attenuates the X-rays which has been emitted from the X-ray tube 111 and of which irradiation range has been limited by the collimator 117.

The DAS 118 acquires count data by counting the number of X-ray photons for each energy band, based on an X-ray detection result by the X-ray detector 112. For example, the DAS 118 includes an amplifier that performs amplification processing on an electrical signal output from each detection element of the X-ray detector 112 and an analog-to-digital (A/D) converter that converts the electrical signal into a digital signal and acquires count data. The DAS 118 is an example of an acquisition unit.

The count data acquired by the DAS 118 is transmitted from a transmitter including a light emitting diode (LED) provided in the rotation frame 113 via optical communication to a receiver including a photodiode provided in a non-rotation part (for example, a fixing frame or the like, which is not illustrated in FIG. 1) of the gantry 110 and then transferred to the console 140. Here, a non-rotation part is, for example, a fixing frame or the like that rotatably supports the rotation frame 113. A method for transmitting data from the rotation frame 113 to the non-rotation part of the gantry 110 is not limited to the optical communication, and any non-contact data transmission method or a contact data transmission method may be adopted.

The bed 130 is a device on which the subject P, which is a scan target, is placed and moved, and includes a base 131, a bed driving device 132, the tabletop 133, and a support frame 134. The base 131 is a housing that supports the support frame 134 to be movable in a vertical direction. The bed driving device 132 is a driving mechanism that moves the tabletop 133, on which the subject P is placed, in a major axis direction of the tabletop 133, and includes a motor and an actuator. The tabletop 133 provided on an upper surface of the support frame 134 is a plate on which the subject P is placed. The bed driving device 132 may move the support frame 134 in the major axis direction of the tabletop 133 in addition to the tabletop 133.

The console 140 includes a memory 141, a display 142, the input interface 143, and processing circuitry 144. While the console 140 is described as a device provided separately from the gantry 110, the gantry 110 may include the console 140 or a part of components of the console 140.

The memory 141 is realized by, for example, a semiconductor memory element, such as a random access memory (RAM) or a flash memory, a hard disk, and an optical disk. The memory 141 stores, for example, count data and image data generated based on the count data. Further, for example, the memory 141 stores a program for causing circuitry included in the photon counting CT apparatus 10 to realize functions of the photon counting CT apparatus 10. The memory 141 may be realized by a cloud.

The display 142 displays various information. For example, the display 142 displays various images generated by the processing circuitry 144 or displays a graphical user interface (GUI) to receive various operations from an operator. For example, the display 142 is a liquid crystal display or a cathode ray tube (CRT) display. The display 142 may be of a desktop type or may be configured with a tablet terminal or the like that wirelessly communicates with a main body of the console 140.

The input interface 143 receives various input operations from the operator, converts the received input operations into electrical signals, and outputs the electrical signals to the processing circuitry 144. For example, the input interface 143 is realized by a mouse, a keyboard, a trackball, a switch, a button, a joystick, a touchpad where an input operation is performed by touching an operation surface, a touchscreen in which a display screen and the touchpad are integrated, non-contact input circuitry using an optical sensor, voice input circuitry, and the like. The input interface 143 may be provided in the gantry 110. Further, the input interface 143 may be configured with a tablet terminal or the like that wirelessly communicates with the main body of the console 140. The input interface 143 is not limited to one equipped with a physical operation component, such as a mouse and a keyboard. For example, an example of the input interface 143 includes electrical signal processing circuitry that receives an electrical signal corresponding to an input operation from an external input device provided separately from the console 140 and outputs the electrical signal to the processing circuitry 144.

The processing circuitry 144 controls entire operations of the photon counting CT apparatus 10 by executing a setting function 144a, a control function 144b, and an output function 144c. The setting function 144a is an example of a setting unit. The control function 144b is an example of a control unit.

For example, the processing circuitry 144 reads a program corresponding to the setting function 144a from the memory 141, executes the program, and thus sets various scan conditions. Examples of scan conditions include a band setting, which is a setting of an energy band in acquiring count data. Details of the band setting is described below.

The processing circuitry 144 reads a program corresponding to the control function 144b from the memory 141, executes the program, and thus executes a scan on the subject P. For example, the control function 144b controls the X-ray high voltage device 114 to supply a high voltage to the X-ray tube 111. As a result, the X-ray tube 111 generates X-rays with which the subject P is to be irradiated. The control function 144b also controls the bed driving device 132 to move the subject P into an imaging port of the gantry 110. Further, the control function 144b adjusts an opening degree and a position of the collimator 117. The control function 144b also controls the control device 115 to rotate the rotation part. While the control function 144b executes a scan, the DAS 118 acquires an X-ray signal from each detection element in the X-ray detector 112 and generates count data.

Further, the control function 144b may execute various types of processing using the count data. For example, the control function 144b performs preprocessing on the count data and performs reconstruction processing on the data subjected to the preprocessing to generate image data (volume data).

For example, the control function 144b performs preprocessing, such as logarithmic transformation processing, offset correction processing, inter-channel sensitivity correction processing, and beam hardening correction on the count data output from the DAS 118. The count data after subjected to preprocessing is also referred to as raw data. The count data before subjected to preprocessing and the raw data after subjected to preprocessing are collectively referred to as projection data. Further, the control function 144b performs reconstruction processing on the projection data using a filtered back projection method or a successive approximation reconstruction method to reconstruct image data. Furthermore, the control function 144b may perform various types of image processing based on the acquired count data, such as material decomposition processing, which is described below.

The processing circuitry 144 reads a program corresponding to the output function 144c from the memory 141, executes the program, and thus outputs various information. For example, the output function 144c controls display on the display 142. Further, for example, the output function 144c transmits various data acquired by executing a scan on the subject P to other devices. For example, the output function 144c transmits the projection data and image data to a picture archiving and communication system (PACS) via a network (not illustrated) and registers the data in the PACS.

In the photon counting CT apparatus 10 illustrated in FIG. 1, each processing function is stored in the memory 141 in a form of a program that is executable by a computer. The processing circuitry 144 is a processor that realizes a function corresponding to each program by reading the program from the memory 141 and executing the program. In other words, the processing circuitry 144 in a state where a program is read out has a function corresponding to the read program.

While, in FIG. 1, it is described that single circuitry, i.e., the processing circuitry 144, realizes the setting function 144a, the control function 144b, and the output function 144c, the processing circuitry 144 may be configured by combining a plurality of independent processors, and each processor may execute a program to realize the functions. Further, each processing function included in the processing circuitry 144 may be appropriately distributed or integrated and realized by a single or a plurality of processing circuitries.

The term “processor” used in the above descriptions means, for example, a CPU, a graphics processing unit (GPU), or circuitry, such as an application specific integrated circuit (ASIC), a programmable logic device (for example, a simple programmable logic device (SPLD), a complex programmable logic device (CPLD)), and a field programmable gate array (FPGA)). The processor realizes the function by reading and executing a program stored in the memory 141.

In FIG. 1, it is described that the single memory 141 stores a program corresponding to each processing function. However, the exemplary embodiment is not limited to this. For example, a configuration may also be adopted in which a plurality of memories 141 is distributed and arranged and the processing circuitry 144 reads the corresponding program from each individual memory 141. Instead of storing the program in the memory 141, the program may be directly incorporated into the circuitry in the processor. In this case, the processor realizes the function by reading and executing the program incorporated in the circuitry.

The processing circuitry 144 may realize the function using a processor of an external apparatus connected via a network NW. For example, the processing circuitry 144 reads and executes a program corresponding to each function from the memory 141 and uses a server group (cloud) connected to the photon counting CT apparatus 10 via the network NW as a calculation resource to realize each function illustrated in FIG. 1.

The overall configuration of the photon counting CT apparatus 10 serving as a PCCT apparatus is described above. Here, various data is able to be generated from the count data by counting the number of X-ray photons for each energy band, but a setting of an appropriate energy band is different depending on an application. Further, converting the count data to add up information of the energy bands is performable, but dividing the added up data into energy bands is not performable. In view of these facts, it can be considered that a large number of energy bands are set in order to be able to generate target data later. However, since a data size increases in accordance with the number of energy bands, it is desirable that the number of energy bands be smaller in consideration of a load of data transmission and storage.

Therefor, the photon counting CT apparatus 10 dynamically changes the number of energy bands by processing that is performed by the processing circuitry 144, to enable acquisition of target data while increase in the data size of the count data is suppressed.

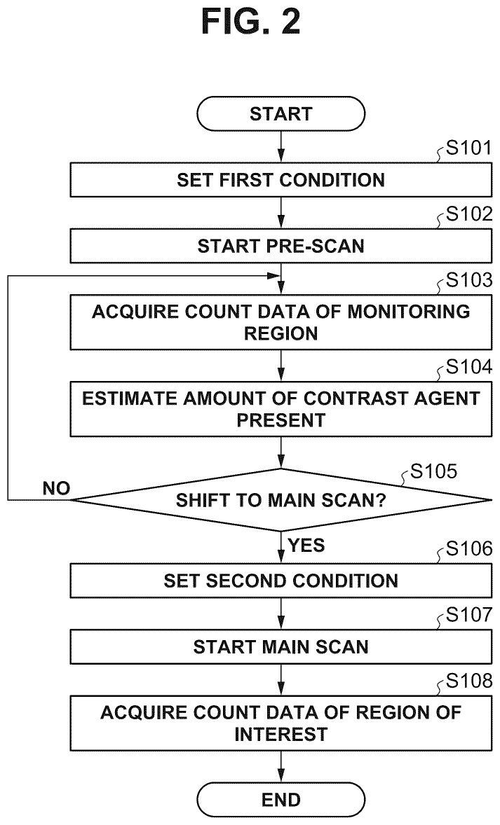

A series of processing performed by the processing circuitry 144 is described below with reference to a flowchart illustrated in FIG. 2. FIG. 2 illustrates a flowchart of a series of processing that is performed by the photon counting CT apparatus according to the first exemplary embodiment. In FIG. 2, a case is described in which a main scan and a pre-scan, which determines a timing to start the main scan, are executed successively.

The main scan is a scan that is performed on a region of interest including a region suspected of having a disease or a region to be treated for a purpose of acquiring a diagnostic image. Here, a contrast agent may be used in the main scan. A contrast agent is injected into the subject P, and the main scan is performed in a state where the region of interest is sufficiently filled with the contrast agent, so that it is possible to acquire a diagnostic image in which the region of interest is contrasted. The main scan is an example of a second scan.

In a case where the main scan is performed using the contrast agent, it is desirable to identify a timing at which the region of interest is filled with the contrast agent, to start the scan. If a large amount of the contrast agent is used, a period during which the region of interest is filled with the contrast agent is longer, and it becomes easy to acquire image data in a state where the region of interest is filled with the contrast agent. However, there is a concern about a side effect on the subject P due to the use of a large amount of the contrast agent. Further, if a CT scan is performed continuously for a long time, it becomes easy to acquire image data in a state where the region of interest is filled with the contrast agent. However, there is a concern about an increase in an amount of radiation exposure to the subject P. From the above, in order to suppress the usage amount of the contrast agent and the amount of radiation exposure to the subject P, it is desirable to identify an appropriate timing at which the region of interest is filled with the contrast agent, to start a scan and to complete the scan in a short time. In view of these facts, in FIG. 2, the pre-scan is performed to determine a timing to start the main scan. The pre-scan is an example of a first scan.

Specifically, in the pre-scan, data is acquired to estimate an amount of contrast agent present in a monitoring region set near the region of interest. For example, in a case where the contrast agent is injected into a blood vessel, the monitoring region is set upstream of the blood flow from the region of interest. By shifting to the main scan at a timing when the contrast agent reaches the monitoring region, diagnostic images are acquired in a state where the region of interest is sufficiently contrasted.

In step S101, the setting function 144a sets a first condition as a setting of the energy band in the pre-scan. The first condition is a condition suitable for generating data to estimate the amount of contrast agent present. For example, in a case of a substance that exhibits a k-edge, such as an iodine-based contrast agent, an enhanced image is generated using K-edge imaging. Thus, the setting function 144a sets two energy bands with a k-edge as a threshold value as the first condition.

In step S102, the control function 144b starts the pre-scan under the first condition set by the setting function 144a. Specifically, the control function 144b causes the X-ray tube 111 to irradiate a range including the monitoring region of the subject P with X-rays. The pre-scan may be performed while the rotation part in the photon counting CT apparatus 10 is rotated or may be performed without rotating the rotation part.

In step S103, while the pre-scan is performed, the DAS 118 acquires count data of the monitoring region under the first condition based on the X-ray detection result by the X-ray detector 112. For example, the DAS 118 acquires count data by counting the number of X-ray photons for each of two energy bands having the k-edge as the threshold value.

In step S104, the control function 144b estimates the amount of contrast agent present in the monitoring region, based on the count data acquired during the pre-scan. In step S105, the control function 144b determines whether to shift to the main scan based on the estimated result of the amount of contrast agent present.

For example, the control function 144b calculates a difference between the count values (count numbers) in the two energy bands set with the k-edge as the threshold value. Here, in a case where the contrast agent flows into the monitoring region and the substance having the k-edge is present in an X-ray path, the difference value between the count values increases. In other words, the difference value is an index indicating the amount of contrast agent present, and, for example, by comparing the difference value with the threshold value, it is possible to determine whether shifting to the main scan is to be performed. The difference value may be acquired based on reconstructed image data or projection data before reconstruction. In other words, reconstruction processing is omittable in determination of whether shifting to the main scan is to be performed.

In a case where shifting to the main scan is not to be performed (NO in step S105), the processing returns to step S103, and acquisition of the count data and estimation of the amount of contrast agent present are continued. On the other hand, in a case where shifting to the main scan is to be performed (YES in step S105), the processing proceeds to step S106. In step S106, the setting function 144a sets a second condition as a setting of the energy band in the main scan. In step S107, the control function 144b starts the main scan under the second condition, and in step S108, acquires count data of the region of interest. A timing for setting the second condition is not limited to the example illustrated in FIG. 2, and the setting may be performed before the processing in step S105. For example, steps S101 and S106 may be integrated, and the first and second conditions may be set before starting the pre-scan.

The second condition is set according to a purpose of the diagnosis. For example, the setting function 144a sets the second condition according to a tissue or a site included in the region of interest. Further, for example, the setting function 144a sets the second condition according to a disease that the subject P is suffering from or is suspected of suffering from. As an example, the setting function 144a sets the second condition in such a manner that a signal-to-noise (SN) ratio of a fatty organ is optimized.

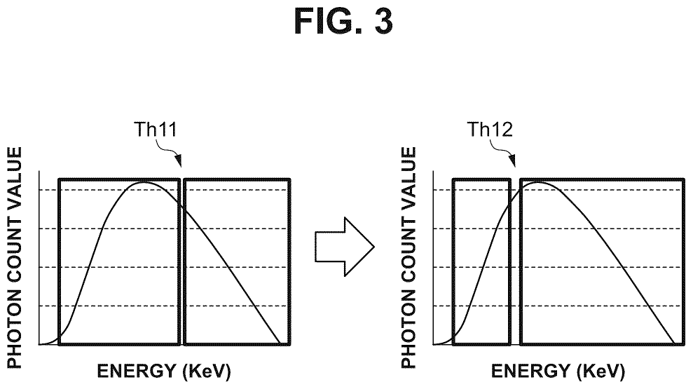

FIG. 3 illustrates a setting example of the first and second conditions. In each graph in FIG. 3, a horizontal axis indicates an X-ray energy (keV), and a vertical axis indicates a photon count value. As illustrated as curves in FIG. 3, the X-rays for use in a CT scan are polychromatic X-rays with an energy range.

A left diagram in FIG. 3 illustrates the setting example of the first condition, in which two energy bands are set with a threshold value Th11 as the bin threshold value. The threshold value Th11 is set, for example, in accordance with a k-edge of a substance contained in the contrast agent.

A right diagram in FIG. 3 illustrates the setting example of the second condition, in which two energy bands are set with a threshold value Th12 as the bin threshold value. For example, in a case of shifting from the pre-scan to the main scan, the control function 144b changes the setting of the energy band as illustrated in FIG. 3. For example, the threshold value Th12 is set in such a manner that data that meets the purpose of the diagnosis is to be generated.

In a case where the main scan is performed by setting a plurality of energy bands, for example, it is possible to optimize a contrast of a specific medium or acquire a functional image, such as K-edge imaging, according to the purpose of the diagnosis. For example, in a case where the two energy bands are set as illustrated in FIG. 3, the control function 144b is able to perform material decomposition processing to estimate an amount of each of two reference materials present. The material decomposition processing is executed by, for example, solving the following Equations (1) and (2).

$$N ( bin 1 ) = N 0 ( bin 1 ) \times exp ( - ( u 1 ( bin 1 ) L 1 + u 2 ( bin 1 ) L 2 ) ) \qquad ( 1 )$$

$$N ( bin 2 ) = N 0 ( bin 2 ) \times exp ( - ( u 1 ( bin 2 ) L 1 + u 2 ( bin 2 ) L 2 ) ) \qquad ( 2 )$$

In Equations (1) and (2), “N(bin1)” is a count value of an energy bin “bin1”. In other words, “N(bin1)” is the number of X-ray photons having X-ray energy included in the energy bin “bin1” among X-ray photons incident on the X-ray detector 112. Similarly, “N(bin2)” is a count value of an energy bin “bin2”. Here, “u1(bin1)” indicates a linear attenuation coefficient of a first reference material in the energy bin “bin1”, “u2(bin1)” indicates a linear attenuation coefficient of a second reference material in the energy bin “bin1”, “u1(bin2)” indicates the linear attenuation coefficient of the first reference material in the energy bin “bin2”, and “u2(bin2)” indicates the linear attenuation coefficient of the second reference material in the energy bin “bin2”. Values of these linear attenuation coefficients are known. “L1” is a path length in which a material 1 exists, and “L2” is a path length in which a material 2 exists. By solving simultaneous equations of Equations (1) and (2), “L1” and “L2” are acquired. The material decomposition processing may be performed on the projection data or on the image data after reconstruction.

The control function 144b generates, for example, a reference material image that highlights the reference material, as a result of the material decomposition processing. For example, in a case where the two energy bands are set, the reference material image that highlights the first reference material and the reference material image that highlights the second reference material are generated. Selection of the reference material can be estimated from the purpose of the diagnosis, and thus the setting function 144a sets the energy band in the main scan in accordance with the reference material. For example, in a case where the disease of the subject P is bone marrow edema, the setting function 144a sets the two energy bands in such a manner that separation capacity of “calcium” and “water” is improved.

Further, the control function 144b also generates various images, such as a virtual monochrome X-ray image (also described as a monochromatic image) at a predetermined energy, a density image, and an effective atomic number image, by performing weighting calculation processing based on a mixing ratio of each reference material using a plurality of reference material images. The output function 144c also displays the various generated image data on the display 142.

As described above, the photon counting CT apparatus 10 according to the first exemplary embodiment includes the X-ray tube 111, the X-ray detector 112, the DAS 118, the setting function 144a, and the control function 144b. The X-ray tube 111 generates X-rays. The X-ray detector 112 is a photon counting type X-ray detector that detects the X-rays that has passed through the subject P. The DAS 118 acquires the count data by counting the number of X-ray photons for each energy band, based on the X-ray detection result by the X-ray detector 112. The setting function 144a sets the first and second conditions as the band settings that are the settings of the energy band. The control function 144b successively executes the first scan under the first condition and the second scan under the second condition on the subject P placed on the tabletop 133. As a result, the photon counting CT apparatus 10 acquires target data while increase in the data size of the count data acquired using the X-ray detector 112 of a photon counting type is suppressed.

For example, as described with reference to FIGS. 2 and 3, the photon counting CT apparatus 10 is able to acquire data according to a purpose of the pre-scan and data according to a purpose of the main scan, respectively. In other words, the photon counting CT apparatus 10 is able to acquire the count data by setting the energy band suitable for determining a degree of accumulation of the contrast agent in the pre-scan and setting the energy band corresponding to the purpose of the diagnosis in the main scan. Further, the number of energy bands set in the pre-scan and the main scan is two, which can be said to be a minimum data size for the count data by the PCCT. In this way, with the photon counting CT apparatus 10, increase in the data size of the count data is suppressed, and a load of data transmission and storage is reduced while image capturing advantageous for the PCCT is performed.

Various benefits can be acquired by suppressing the data size of the count data. First, by reducing amounts of storage and lines that are used, a cost related to data management are expected to be reduced. Further, by suppressing increase in the data size, a time required for transmitting and processing the count data is shortened and a real time nature of processing based on the count data is improved. For example, by suppressing increase in the data size of the count data acquired in the pre-scan, the processing for estimating the amount of contrast agent present is accelerated, whereby a timing for shifting to the main scan is more accurately determined.

The number of energy bands is not the only parameter that affects the data size of the count data. For example, a pixel size, the number of views, a dynamic range, and the like also affect the data size of the count data.

The pixel size is a condition for binning in the detection element of the X-ray detector 112. Binning is processing in which a plurality of detection elements (detection element group) in the X-ray detector 112 is treated as one pixel, and signals acquired from the detection element group are bundled into one signal and treated. With an increase in the number of the detection elements included in the detection element group, the data size of the count data decreases. The number of views is the number of data in a time direction. For example, with increase in frequency in which the DAS 118 reads a signal form the X-ray detector 112, the number of views increases and the data size of the count data increases. The dynamic range is a data range assigned to each individual count value. For example, in a case where the dynamic range is excessively small and a high dose of X-rays is incident, the count value may overflow, and the count data may be improperly acquired. It is desirable to set the dynamic range in such a manner that overflow does not occur, but with increase in the dynamic range, the data size of the count data increases.

The photon counting CT apparatus 10 may offset increase in the data size due to reducing the pixel size, increasing the number of views, or widening the dynamic range, with a reduction in the data size by reducing the number of energy bands. In other words, according to the above-described exemplary embodiment, the photon counting CT apparatus 10 may reduce the data size of the count data or may adjust various scan conditions while increase in the data size is suppressed.

FIG. 3 illustrates an example in which, in shifting from the pre-scan to the main scan, only the threshold value between the energy bands is changed while the number of energy bands is maintained. However, the exemplary embodiment is not limited to this, and the number of energy bands may also be changed.

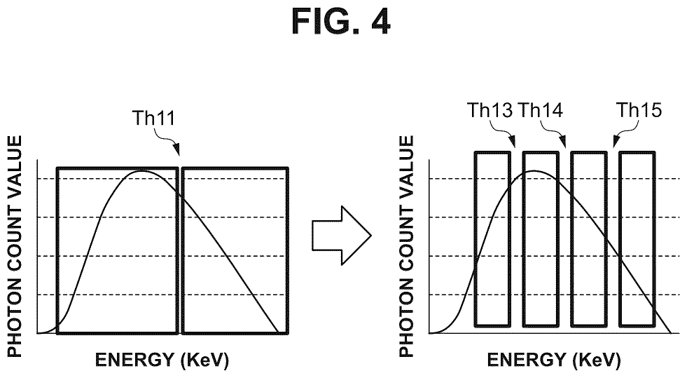

For example, the setting function 144a sets four energy bands with threshold values Th13, Th14, and Th15 between the energy bands, respectively, as the second condition as illustrated in a right diagram in FIG. 4. In other words, the photon counting CT apparatus 10 acquires information in detail only in the main scan in such a manner that information to be used is ensured. In this case, although the data size of the count data acquired in the main scan increases, acquiring information to be used for diagnosis is necessary. Further, even in the case illustrated in FIG. 4, only two energy bands are set in the pre-scan, and the data size of the count data is suppressed.

### Second Exemplary Embodiment

According to the above-described first exemplary embodiment, the case in which the pre-scan and the main scan are successively executed is described as an example. However, the exemplary embodiment is not limited to this and may be similarly applied to another case in which a plurality of scans is successively executed on the subject P.

A photon counting CT apparatus 10 according to a second exemplary embodiment includes a similar configuration to that of the photon counting CT apparatus 10 illustrated in FIG. 1, and some processing by a setting function 144a and a control function 144b is different. Hereinbelow, the points described in the first exemplary embodiment are denoted by the same reference numerals in FIG. 1, and the redundant descriptions are omitted.

For example, there may be a case in which a plurality of regions of interest is set for the subject P, and a diagnostic image is acquired for each of the regions of interest. Alternatively, there may be a case in which a plurality of diagnostic images is acquired for one region of interest under different scan conditions. In other words, there may be a case in which a plurality of main scans is successively executed. In this case, the setting function 144a sets the first condition according to the purpose of the diagnosis in the first scan and sets the second condition according to the purpose of the diagnosis in the second scan.

Before the main scan, a positioning scan may be executed in some cases to determine a scan range in the main scan. The positioning scan is also referred to as scanography and scout imaging. In this case, the setting function 144a sets the first condition in such a manner that data suitable for positioning processing is acquired, and sets the second condition according to the purpose of the diagnosis. For example, in a case where the purpose of the main scan is to acquire a diagnostic image relating to a specific organ, the setting function 144a is able to improve accuracy of the positioning processing by setting the first condition to optimize a contrast of the specific organ.

The first and second conditions may be different only in the threshold values between the energy bands or may be different in the number of energy bands. For example, in a case where the first scan is more important than the second scan, the number of energy bands in the first condition may be set to be larger.

Each of a plurality of scans successively executed may be a full scan that acquires projection data for a 360° view range or may be a half scan that acquires projection data for a view range that is 180° added to an X-ray fan angle. The fan angle is an angle indicating broadening of X-rays emitted from the X-ray tube 111 and detected by the X-ray detector 112. Specifically, as illustrated by dotted lines in FIG. 1, X-rays emitted from the X-ray tube 111 has a fan shape broadening on an XY plane (axial plane of the subject P), and the fan angle corresponds to the central angle of the fan shape. There is no particular limitation on a specific value of the fan angle in performing a half scan, but the fan angle is often about 45°. In this case, half reconstruction can be executed by acquiring projection data for a view range of about 225°.

In the various examples described in the first and second exemplary embodiments, switching between the first and second conditions may be performed for each rotation of the rotation part or may be performed in units of one or a plurality of views. For example, in a case where a half scan is performed as the first scan, it is possible to shift to the second scan at a timing when data of the view range necessary for half reconstruction is acquired without waiting for the rotation part to make one rotation.

### Third Exemplary Embodiment

According to the above-described first and second exemplary embodiments, examples are described in which a plurality of scans is successively executed on the subject P placed on the tabletop 133, and the energy band is set for each scan. However, the exemplary embodiments are not limited to this. According to a third exemplary embodiment, an example is described in which a plurality of energy bands is set for one scan.

A photon counting CT apparatus 10 according to the third exemplary embodiment includes a similar configuration to that of the photon counting CT apparatus 10 illustrated in FIG. 1, and some processing by a setting function 144a and a control function 144b is different. Hereinbelow, the points described in the first and second exemplary embodiments are denoted by the same reference numerals in FIG. 1, and the redundant descriptions are omitted.

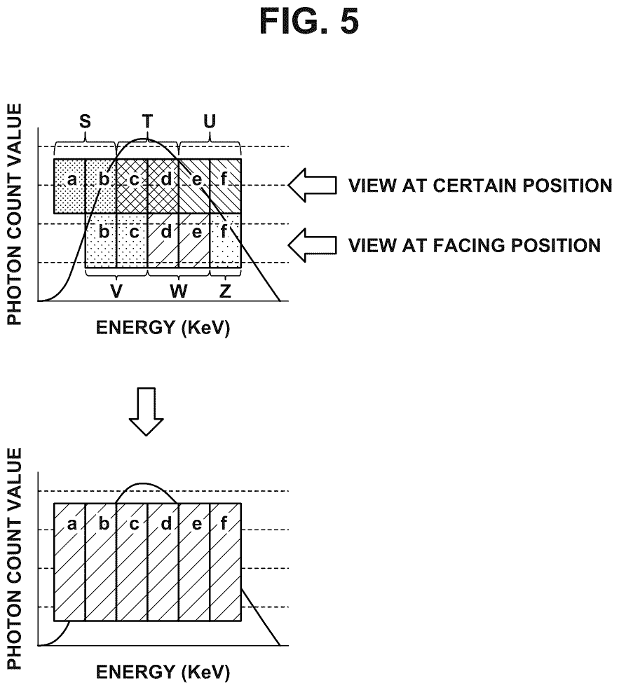

Specifically, the setting function 144a sets a reference condition and a facing condition as settings of the energy band in one scan. The reference condition and the facing condition are described with reference to FIG. 5. FIG. 5 illustrates an example of the band setting according to the third exemplary embodiment. In FIG. 5, energy bands S, T, and U are set as the reference conditions. Further, energy bands V, W, and Z are set as the facing conditions.

Hereinbelow, a certain view is described as a first view, and a view at a position facing the first view (a position 180° opposite) is described as a second view. In a case where the reference condition is set for the first view, the facing condition is set for the second view. For example, the control function 144b executes a scan in such a manner that the reference condition is set in the first view and the facing condition is set in the second view by switching between the reference condition and the facing condition every time the rotation part rotates 180°. Alternatively, the control function 144b executes a scan in such a manner that the reference condition is set in the first view and the facing condition is set in the second view by switching between the reference condition and the facing condition for one or a plurality of views.

Here, when the first and second views are compared, X-ray irradiation directions are opposite, but paths through which X-rays pass (X-ray paths) overlap. Therefore, it is possible to acquire more detailed information about the energy band by performing post-processing on the count data acquired in the first and second views.

Specifically, the energy bands illustrated in FIG. 5 is able to be divided into six smaller energy bands (energy band a, energy band b, energy band c, energy band d, energy band e, and energy band f). The count values in the energy bands S to U are already known from the count data acquired in the first view, and the count values in the energy bands V to Z are already known from the count data acquired in the second view, but count values in the energy bands a to f are unknown.

Here, a total value of the count value in the energy band a and the count value in the energy band b is equal to the count value in the energy band S. In other words, a relationship of the energy band a and the energy band b to the energy band S is expressed by an equation. Similarly, a relationship of the energy band c and the energy band d to the energy band T is expressed by an equation. Similarly, a relationship of the energy band e and the energy band f to the energy band U is expressed by an equation. Similarly, a relationship of the energy band b and the energy band c to the energy band V is expressed by an equation. Similarly, a relationship of the energy band d and the energy band e to the energy band W is expressed by an equation. Similarly, a relationship of the energy band f to the energy band Z is expressed by an equation. In this way, six equations are set in the example illustrated in FIG. 5, and there are six unknowns, so that each of the count values in the energy bands a to f are calculated by solving the simultaneous equations.

As described above, in the example illustrated in FIG. 5, the number of energy bands of the count data that is acquired in each view is three (energy bands S to U or energy bands V to Z), but it is still possible to acquire a count value of each of six energy bands (energy bands a to f) by post-processing. In other words, with the photon counting CT apparatus 10 according to the third exemplary embodiment, target data is able to be acquired by post-processing as appropriate while increase in the data size of the count data to be acquired is suppressed.

The processing according to the third exemplary embodiment may be executed in combination with the processing according to the first and second exemplary embodiments or may be executed separately. For example, in a case where a plurality of scans is successively executed, the control function 144b executes a part or all of the plurality of scans in such a manner that the reference condition is set in the first view and the facing condition is set in the second view. Alternatively, in a case where only one scan is executed, the control function 144b executes the one scan in such a manner that the reference condition is set in the first view and the facing condition is set in the second view.

### Fourth Exemplary Embodiment

According to the above-described third exemplary embodiment, an example in which the reference condition and the facing condition are set is described as a case in which a plurality of energy bands is set for one scan. According to a fourth exemplary embodiment, another example is described in which a plurality of energy bands is set for one scan.

A photon counting CT apparatus 10 according to the fourth exemplary embodiment includes a similar configuration to that of the photon counting CT apparatus 10 illustrated in FIG. 1, and some processing by a setting function 144a and a control function 144b is different. Hereinbelow, the points described in the first to third exemplary embodiments are denoted by the same reference numerals in FIG. 1, and the redundant descriptions are omitted.

Even in a case where only one scan is performed, there may be a case in which a plurality of targets of interest is set. For example, there is a case where a plurality of types of contrast agents is injected into the subject P, and a degree of accumulation of each agent is a target of interest. Examples of such a case include a case where a first contrast agent that is iodine-based and a second contrast agent that is gadolinium-based are injected into the subject P.

In this case, the setting function 144a sets a first contrast agent condition according to the first contrast agent and a second contrast agent condition according to the second contrast agent, respectively. Further, the control function 144b executes a scan on the subject P while switching between the first contrast agent condition and the second contrast agent condition.

For example, the control function 144b executes a scan while switching between the first contrast agent condition and the second contrast agent condition every time the rotation part rotates 180°. Further, for example, the control function 144b executes a scan while switching between the first contrast agent condition and the second contrast agent condition for one or a plurality of views. The control function 144b may switch between the first contrast agent condition and the second contrast agent condition in such a manner that the first contrast agent condition is set in the first view and the second contrast agent condition is set in the second view at a position facing the first view.

In the foregoing exemplary embodiments, the components of the devices illustrated in the diagrams are functional concepts and do not necessarily need to be physically configured as illustrated in the diagrams. In other words, the specific forms of distribution or integration of the devices are not limited to the illustrated ones, and all or part of the devices may be functionally or physically distributed or integrated into any units depending on various loads and usages. All or part of the processing functions for the devices to perform can be implemented by a CPU and programs to be analyzed and executed by the CPU, or as wired logic hardware.

The method described in the foregoing exemplary embodiments can be implemented by executing a pre-prepared program on a computer, such as a personal computer or workstation. This program can be distributed via a network, such as the Internet. The program may be recorded on a non-transitory computer-readable recording medium, such as a hard disk, a flexible disk (FD), a compact disk read only memory (CD-ROM), a magneto-optical-disc (MO), or a digital versatile disc (DVD), and executed by being read from the recording medium by a computer.

According to at least one of the above-described exemplary embodiments, it is possible to acquire target data while increase in a data size of count data to be acquired using a photon counting type X-ray detector is suppressed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A photon counting computed tomography (CT) apparatus comprising:
an X-ray generator configured to generate an X-ray;
an X-ray detector of a photon counting type configured to detect the X-ray that has passed through a subject; and
processing circuitry configured to
acquire count data by counting the number of X-ray photons for each energy band, based on a detection result of the X-ray by the X-ray detector,
set a first condition and a second condition as band settings that are settings of the energy band, and
execute successively a first scan that is performed under the first condition, and a second scan that is performed under the second condition, on the subject placed on a tabletop.

2. The photon counting CT apparatus according to claim 1,
wherein the first scan is a pre-scan to determine a timing to start the second scan,
wherein the second scan is a main scan to acquire a diagnostic image in which a region of interest in the subject is contrasted using a contrast agent,
wherein the processing circuitry sets a condition in accordance with a type of the contrast agent as the first condition, and sets a condition in accordance with a diagnostic purpose for the region of interest as the second condition, and
wherein the processing circuitry determines the timing by detecting the contrast agent, based on projection data acquired in the first scan, and successively executes the first scan and the second scan.

3. The photon counting CT apparatus according to claim 2, wherein the processing circuitry sets, as the second condition, a condition in which the number of the energy bands is greater than the number of the energy bands in the first condition.

4. The photon counting CT apparatus according to claim 1,
wherein the processing circuitry sets a reference condition and a facing condition as the band setting, and
wherein the processing circuitry executes a scan in such a manner that the reference condition is set in a first view and the facing condition is set in a second view at a position facing the first view, in at least one of the first scan and the second scan.

5. The photon counting CT apparatus according to claim 1, wherein the processing circuitry successively executes the first scan and the second scan by switching between the first condition and the second condition in a view range that is 180° added to a fan angle of the X-ray.

6. The photon counting CT apparatus according to claim 1,
wherein the processing circuitry sets, as the band setting, a first contrast agent condition corresponding to a first contrast agent injected into a body of the subject and a second contrast agent condition corresponding to a second contrast agent injected into the body of the subject, and
wherein the processing circuitry executes a scan while switching between the first contrast agent condition and the second contrast agent condition in at least one of the first scan and the second scan.
